(19) United States
(12) Patent Application Publication
(10) Pub. No.: US 2024/0059622 A1
(43) Pub. Date: Feb. 22, 2024

(54) Coatings for Ceramic Substrates

(71) Applicant: Cardiac Pacemakers, Inc., St Paul, MN (US)

(72) Inventors: Matthew P. Jones, Shoreview, MN (US); Sverre Gropen, Minneapolis, MN (US); Jean M. Bobgan, Maple Grove, MN (US); Keith R. Maile, New Brighton, MN (US)

(21) Appl. No.: 18/226,595

(22) Filed: Jul. 26, 2023

Related U.S. Application Data
(60) Provisional application No. 63/399,297, filed on Aug. 19, 2022.

(51) Int. Cl. C04B 41/90; C04B 35/48; C04B 41/88; C04B 41/87; C04B 41/52; C04B 41/50; C04B 41/51; C04B 41/45; A61N 1/375

(52) U.S. Cl. CPC C04B 41/90; C04B 35/48; C04B 41/88; C04B 41/87; C04B 41/522; C04B 41/5027; C04B 41/5062; C04B 41/5057; C04B 41/5133; C04B 41/4533; C04B 41/4531; A61N 1/3754

(57) ABSTRACT

A method of metallizing a ceramic substrate includes depositing a barrier layer onto the substrate, depositing a tie layer onto the barrier layer, and depositing a metal layer onto the tie layer to metallize the substrate. The barrier layer may include an oxygen rich material, a nitrogen rich material, or a carbon rich material.

### CROSS REFERENCE TO RELATED APPLICATION

This application claims priority to Provisional Application No. 63/399,297, filed Aug. 19, 2022, which is herein incorporated by reference in its entirety.

### TECHNICAL FIELD

The present disclosure relates generally to metallized substrates and methods for making the metallized substrates. More specifically, the present disclosure relates to substrates comprising ceramics such as zirconia and methods and materials for metallizing zirconia-based materials. Such approaches may be useful in medical device applications, among others.

### SUMMARY

In Example 1, a method of metallizing a substrate includes depositing a barrier layer onto the substrate; depositing a tie layer onto the barrier layer; and depositing a metal layer onto the tie layer to metallize the substrate. The barrier layer includes an oxygen rich material, a nitrogen rich material, or a carbon rich material.

In Example 2, the method of Example 1, wherein the substrate includes a ceramic material.

In Example 3, the method of Examples 1 or 2, wherein the dielectric substrate is selected from a group consisting of zirconia, stabilized zirconia, partially stabilized zirconia, tetragonal zirconia, magnesia stabilized zirconia, ceria-stabilized zirconia, yttria stabilized zirconia, and calcia stabilized zirconia.

In Example 4, the method of any of Examples 1 to 3, wherein the oxygen rich material is selected from a group consisting of aluminum oxide, titanium oxide, silicon dioxide, tantalum oxide, niobium oxide, tungsten oxide, and molybenum oxide.

In Example 5, the method of any of Examples 1 to 4, wherein the oxygen rich material has an oxygen percentage of 5% to 80%.

In Example 6, the method of any of Examples 1 to 5, wherein the nitrogen rich material is selected from a group consisting of titanium nitride, aluminum nitride, tantalum nitride, tungsten nitride, niobium nitride, and molybdenum nitride.

In Example 7, the method of any of Examples 1 to 6, wherein the nitrogen rich material has a nitrogen percentage of 5% to 80%.

In Example 8, the method of any of Examples 1 to 7, wherein the carbon rich material is selected from a group consisting of silicon carbide, tungsten carbide, aluminum carbide, tantalum carbide, and molybdenum carbide.

In Example 9, the method of any of Examples 1 to 8, wherein the carbon rich material has a carbon percentage of 5% to 80%.

In Example 10, the method of any of Examples 1 to 9, wherein the tie layer comprises titanium, chromium, molybdenum, niobium, tungsten, tantalum, or vanadium.

In Example 11, the method of any of Examples 1 to 10, wherein the barrier layer is deposited onto the substrate by sputtering, thermal evaporation, or flame spray.

In Example 12, the method of any of Examples 1 to 11, wherein the metal layer allows for brazing the substrate.

In Example 13, the method of any of Examples 1 to 12, wherein a thickness of the barrier layer is from 0.0005 to 50 microns.

In Example 14, a metallized ceramic substrate includes a base ceramic substrate containing zirconium; a barrier layer directly contacting the base substrate; a tie layer directly contacting the barrier layer; and a metal layer coupled to the tie layer.

In Example 15, the ceramic substrate of Example 14, wherein the metal layer is a brazing material.

In Example 16, a method of metallizing a dielectric substrate includes depositing a barrier layer onto the dielectric substrate; depositing a tie layer onto the barrier layer; and depositing a metal layer onto the tie layer to metallize the dielectric substrate. The barrier layer includes an oxygen rich material, a nitrogen rich material, or a carbon rich material.

In Example 17, the method of Example 16, wherein the dielectric substrate includes a ceramic material.

In Example 18, the method of Example 16, wherein the dielectric substrate is selected from a group consisting of zirconia, stabilized zirconia, partially stabilized zirconia, tetragonal zirconia, magnesia stabilized zirconia, ceria-stabilized zirconia, yttria stabilized zirconia, and calcia stabilized zirconia, as well as alumina, titania, and the like.

In Example 19, the method of Example 16, wherein the oxygen rich material is selected from a group consisting of aluminum oxide, titanium oxide, silicon dioxide, tantalum oxide, niobium oxide, tungsten oxide, and molybenum oxide.

In Example 20, the method of Example 16, wherein the oxygen rich material has an oxygen percentage of 5% to 80%.

In Example 21, the method of Example 16, wherein the nitrogen rich material is selected from a group consisting of titanium nitride, aluminum nitride, tantalum nitride, tungsten nitride, niobium nitride, and molybdenum nitride.

In Example 22, the method of Example 16, wherein the nitrogen rich material has a nitrogen percentage of 5% to 80%.

In Example 23, the method of Example 16, wherein the carbon rich material is selected from a group consisting of silicon carbide, tungsten carbide, aluminum carbide, tantalum carbide, and molybdenum carbide.

In Example 24, the method of Example 16, wherein the carbon rich material has a carbon percentage of 5% to 80%.

In Example 25, the method of Example 16, wherein the tie layer comprises titanium, chromium, molybdenum, niobium, tungsten, tantalum, or vanadium.

In Example 26, the method of Example 16, wherein the barrier layer is deposited onto the dielectric substrate by sputtering, thermal evaporation, or flame spray.

In Example 27, the method of Example 16, wherein the metal layer allows for brazing the dielectric substrate.

In Example 28, the method of Example 16, wherein a thickness of the barrier layer is from 0.0005 to 50 microns.

In Example 29, a metallized ceramic substrate includes a base ceramic substrate containing zirconium; a barrier layer directly contacting the base substrate; a tie layer directly contacting the barrier layer; and a metallic layer coupled to the tie layer.

In Example 30, the ceramic substrate of claim 29, wherein the metallic layer is a brazing material.

In Example 31, an implantable medical device having a feed-through assembly includes a metallized ceramic substrate. The metallized ceramic substrate includes a base ceramic substrate containing zirconium; a barrier layer directly contacting the base substrate; a tie layer directly contacting the barrier layer; and a metallic layer coupled to the tie layer.

In Example 32, the implantable medical device of Example 31, further including one or more wires bonded to the metallized ceramic substrate.

In Example 33, the implantable medical device of Example 31, wherein the barrier layer comprises aluminum oxide.

In Example 34, the implantable medical device of Example 31, wherein the dielectric substrate is yttria stabilized zirconia.

In Example 35, the implantable medical device of Example 31, wherein the tie layer comprises titanium.

While multiple instances are disclosed, still other instances of the present disclosure will become apparent to those skilled in the art from the following detailed description, which shows and describes illustrative instances of the disclosure. Accordingly, the drawings and detailed description are to be regarded as illustrative in nature and not restrictive.

### BRIEF DESCRIPTION OF THE DRAWINGS

FIG. 1 is a schematic illustration depicting a patient monitoring system, in accordance with certain instances of the present disclosure.

FIG. 2 illustrates an implantable medical device, in accordance with certain instances of the present disclosure.

FIG. 3 is a perspective view of the implantable medical device of FIG. 2, in accordance with certain instances of the present disclosure.

FIG. 4 is a schematic illustration of an implantable system implanted within a patient that delivers electrical simulation to the heart, in accordance with certain instances of the present disclosure.

FIG. 5 is a schematic illustration of the implantable system of FIG. 4 with a partial cross-sectional view of a pulse generator, in accordance with certain instances of the present disclosure.

FIG. 6A is a perspective view of a feed-thru assembly including a base substrate, in accordance with certain instances of the present disclosure.

FIG. 6B is a perspective front view of the base substrate of FIG. 6A.

FIG. 6C is a blown-up cross-sectional view of the surface of the base substrate of FIG. 6A.

FIGS. 7A-7H are schematic illustrations of an assembly process of an exemplary feed-thru assembly, in accordance with certain instances of the present disclosure.

FIG. 8 shows a schematic of a structure containing a substrate, a barrier layer, a tie layer, and a metal layer, in accordance with certain instances of the present disclosure.

FIG. 9 is a flowchart illustrating a method of metallizing a dielectric substrate, in accordance with certain instances of the present disclosure.

While the disclosure is amenable to various modifications and alternative forms, specific instances have been shown by way of example in the drawings and are described in detail below. The intention, however, is not to limit the disclosure to the particular instances described. On the contrary, the disclosure is intended to cover all modifications, equivalents, and alternatives falling within the scope of the disclosure as defined by the appended claims.

### DETAILED DESCRIPTION

Ceramic materials are useful in many applications including medical device applications because of their biocompatibility in addition to benefits such as pass-through signal transmission (e.g., electromagnetic signal transparency), structural integrity (e.g., corrosion resistance, structural strength), and/or ability to be hermetically sealed. Such materials are useful for various components in medical devices such as therapy devices (e.g., pacemakers, defibrillators, neuromodulation devices, ablation needles, ablation catheters, rechargeable IMDs, puncture and cautery devices) and sensing devices.

Zirconia based ceramic materials are particularly useful due to their strength and durability. However, zirconia based ceramics have limitations when metallized using physical vapor deposition (“PVD”) processes (e.g., sputter deposition, thermal evaporation, cathodic arc deposition, electron-beam physical vapor deposition, evaporative deposition, close-space sublimation, pulsed laser deposition, pulsed electron deposition, and sublimation sandwich methods). To metallize ceramic materials such as zirconia based materials, a tie layer can be deposited between a ceramic substrate and a metal layer. However, depositing tie layer materials onto substates containing zirconia based ceramics can cause breakdown and erosion of the material of substrates—in particular at and near the interface of the substrate and tie layer. Because the substrates may contain elements such as oxygen, nitrogen, and/or carbon and the tie layers contain materials that are “hungry” for such elements, the tie layer can essentially “eat” or consume such elements which degrades the material and structure of the substrate. Instances of the present disclosure help address such issues.

In certain instances, a barrier layer is deposited between the substrate and the tie layer to serve as a buffer between the oxygen-hungry, nitrogen-hungry, and/or carbon-hungry tie layer. The barrier layer can comprise a material that is rich in oxygen, nitrogen, and/or carbon such that the barrier layer can afford to lose such elements to the hungry tie layer. As such, the substrate is less likely to degrade.

In other instances, the tie layer itself—or at least a portion thereof closest to the substrate—may be formulated to comprise higher-than-typical amount of oxygen, nitrogen, and/or carbon and/or by comprising a lower-than-typical amount of metal (e.g., metals such as titanium, chrome, tantalum, tungsten, molybdenum, niobium, or nickel). As such, the tie layer be less hungry for the substrate's oxygen, nitrogen, and/or carbon.

The approaches described herein have many applications. Certain medical device applications are described below, but the approaches can be used for applications outside of medical devices and for medical devices that are not expressly shown in one of the Figures.

In medical device applications, certain ceramic components may need to be metallized. Metallization can be used as conductive pathways for electrical signals, for conductive pathways for heat, to be reflective for optical applications, and/or to provide a bonding surface for adhesives, welding, soldering, and/or brazing.

### Exemplary Implantable Medical Device Applications

### Example 1

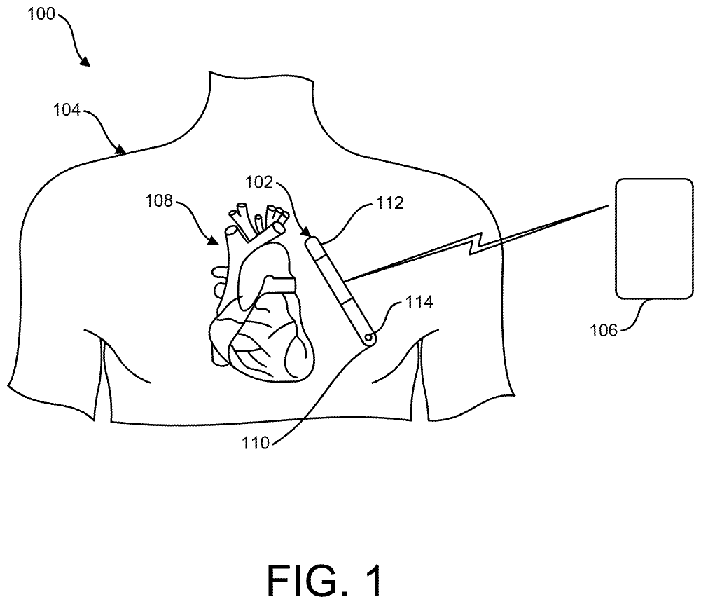

FIG. 1 is a schematic illustration of a system 100 including an implantable medical device (IMD) 102 implanted within a patient's body 104 and configured to communicate with a receiving device 106. Components of the IMD 102 such as the outer housing and the electrical feed-thru assembly can comprise a zirconia-containing material—portions of which are metallized and comprise a barrier layer described further below.

In instances, the IMD 102 may be implanted subcutaneously within an implantation location or pocket in the patient's chest or abdomen and may be configured to monitor (e.g., sense and/or record) physiological parameters associated with the patient's heart 108. The IMD 102 may be an implantable cardiac monitor (ICM) (e.g., an implantable diagnostic monitor (IDM), an implantable loop recorder (ILR)) configured to record physiological parameters such as, for example, one or more cardiac activation signals, heart sounds, blood pressure measurements, oxygen saturations, and/or the like. For example, the IMD 102 may include sensors or circuitry for detecting respiratory system signals, cardiac system signals, and/or signals related to patient activity. In instances, the IMD 102 may be configured to sense intrathoracic impedance, from which various respiratory parameters may be derived, including, for example, respiratory tidal volume and minute ventilation. In instances, the IMD 102 may be configured to monitor physiological parameters that may include one or more signals indicative of a patient's physical activity level and/or metabolic level, such as an acceleration signal. Sensors and associated circuitry may be incorporated in connection with the IMD 102 for detecting one or more body movement or body posture and/or position related signals. For example, accelerometers and/or GPS devices may be employed to detect patient activity, patient location, body orientation, and/or torso position. The IMD 102 may be configured to sense and/or record at regular intervals, continuously, and/or in response to a detected event.

As shown, the IMD 102 may include a housing 110 having two electrodes 112 and 114 coupled thereto. According to instances, the IMD 102 may include any number of electrodes (and/or other types of sensors such as thermometers, barometers, pressure sensors, optical sensors, motion sensors, and/or the like) in any number of various types of configurations, and the housing 110 may include any number of different shapes, sizes, and/or features. In instances, the IMD 102 may be configured to sense physiological parameters and record the physiological parameters. For example, the IMD 102 may be configured to activate (e.g., periodically, continuously, upon detection of an event, and/or the like), record a specified amount of data (e.g., physiological parameters) in a memory, and communicate that recorded data to the receiving device 106. In the case of an IDM, for example, the IMD 102 may activate, record cardiac signals for a certain period of time, deactivate, and activate to communicate the recorded signals to the receiving device 106. In various instances, the receiving device 106 may be, for example, a programmer, controller, patient monitoring system, and/or the like.

The IMD 102 may include a header that may house various circuitry components within its interior such as an antenna. In instances, the IMD 102 and the receiving device 106 may communicate through a wireless link. For example, the IMD 102 and the receiving device 106 may be coupled through a short-range radio link, such as Bluetooth, IEEE 802.11, and/or a proprietary wireless protocol. The communications link may facilitate uni-directional and/or bi-directional communication between the IMD 102 and the receiving device 106. Data and/or control signals may be transmitted between the IMD 102 and the receiving device 106 to coordinate the functions of the IMD 102 and/or the receiving device 106. In instances, patient data may be downloaded from one or more of the IMD 102 and the receiving device 106 periodically or on command. The physician and/or the patient may communicate with the IMD 102 and the receiving device 106, for example, to acquire patient data or to initiate, terminate, or modify recording and/or therapy.

In some instances, the housing 110 may be a core assembly housing having controller circuitry enclosed within. The controller circuitry is coupled, at the first end, to a first feed-thru assembly, and coupled, at the second end, to a second feed-thru assembly. The feed-thru assembly may be configured to provide a pathway for electrical connections between the circuitry components of the header (e.g., the electrode and the antenna) to the controller circuitry. Similarly, the second feed-thru assembly may be configured to provide a pathway for electrical connections between one or more batteries and/or the electrode to the controller circuitry.

As will be discussed in more details below, portions of the housing and/or the feed-thru assemblies may include a metallized dielectric substrate with a barrier layer (e.g., coating) to protect the underlying dielectric substrate.

### Example 2

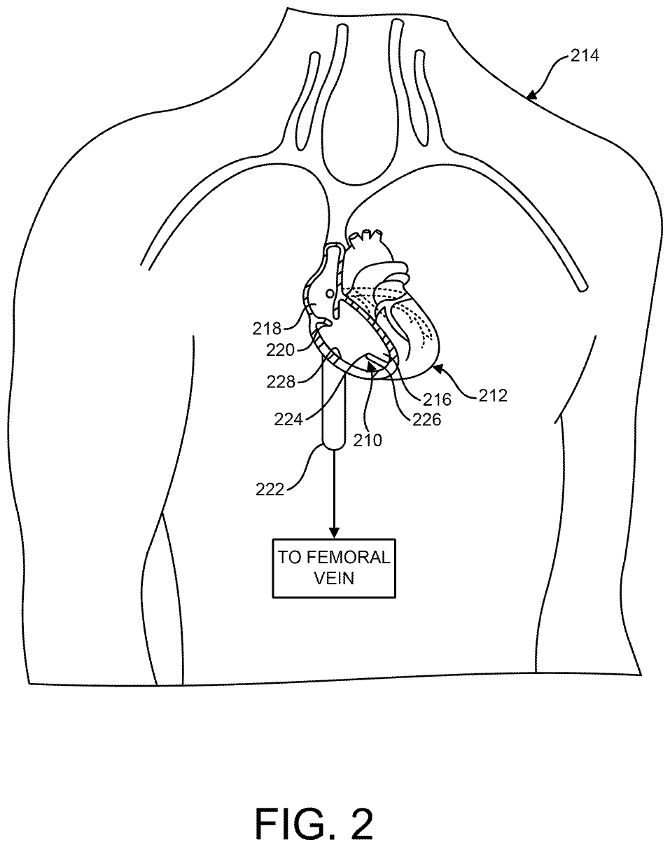

FIG. 2 illustrates an implantable medical device (IMD) 210 in the form of a leadless cardiac pacemaker (LCP) implanted in a heart 212 of a patient 214. Components of the IMD 210 such as the outer housing and the electrical feed-thru assembly can comprise a zirconia-containing material—portions of which are metallized and comprise a barrier layer described further below.

As shown in FIG. 2, the heart 212 includes a right ventricle 216, a right atrium 218, and a tricuspid valve 220 separating the right atrium 218 from the right ventricle 216. An inferior vena cava 222 leads to the right atrium 218. The IMD 210 may be implanted in the right ventricle 216. The IMD 210 may have a proximal end 224 and a distal end 226. The IMD 210 may be implanted such that the proximal end 224 is nearest the tricuspid valve 220 and the distal end 226 is in contact with an endocardium 228 lining the walls of the right ventricle 216. Once the IMD 210 is implanted, the IMD 210 can provide electrophysiological therapy to the heart 212.

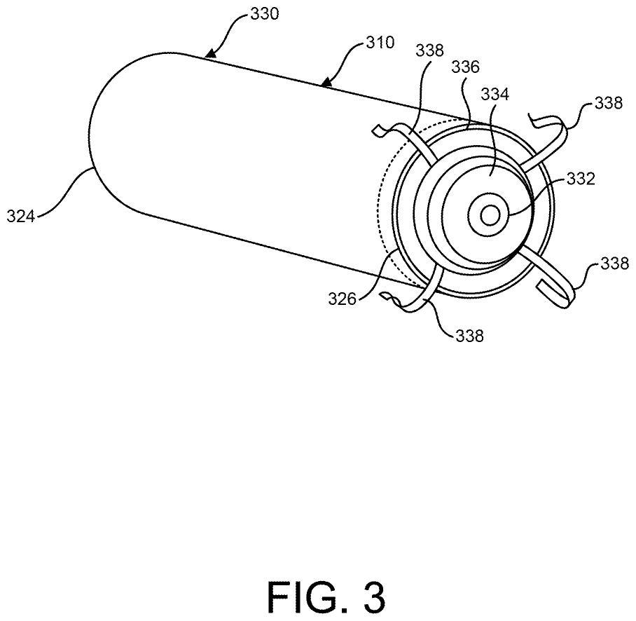

FIG. 3 is a perspective view of an implantable medical device of FIG. 2. In the instance shown in FIG. 3, the IMD 310 includes a case 330, an electrode 332, an electrode insulator 334, a case insulator 336, and a plurality of tines 338 (four shown). The case 330 has a roughly cylindrical shape and extends from the proximal end 324 to the distal end 326. The case 330 may house control and communication electronics and a battery. The case 330 may be formed of a biocompatible metal, for example, a zirconia-containing material as described in more detail below. In some instances, the electrode 332 may be formed of a biocompatible metal, for example, titanium, iridium, gold, or stainless steel. The plurality of tines 338 may be formed of a flexible, resilient, biocompatible metal, for example, nitinol. The electrode 332 and the plurality of tines 338 are disposed at the distal end 326. The electrode insulator 334 is disposed between the electrode 332 and the plurality of tines 38 to electrically insulate the electrode 332 from the plurality of tines 338. The case insulator 336 is disposed between the case 330 and the tines 338 to electrically insulate the case 330 from the tines 338.

As will be discussed in more details below; the case 330, the electrode insulator 334, and/or the case insulator 336 may be formed of a metallized dielectric substrate with a barrier layer (e.g., coating) to protect the underlying dielectric substrate.

### Example 3

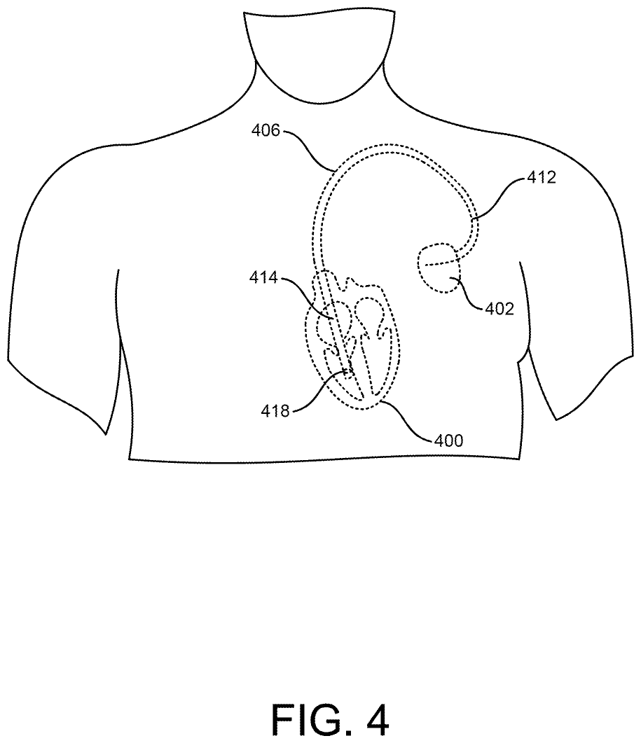

FIG. 4 is a schematic illustration of an implantable system within a patient that delivers electrical simulation to the heart. Components of the pulse generator 402 such as the outer housing and the electrical feed-thru assembly can comprise a zirconia-containing material—portions of which are metallized and comprise a barrier layer described further below.

Although FIG. 4 is shown delivering electrical simulation to the heart, instances of the present disclosure can also be used in devices where electrical stimulation is provided to other areas, for example, spinal cord stimulation, as well as subcutaneous electrode configurations. The instance of FIG. 4 shows that the implantable system includes a pulse generator 402 arranged for producing electrical stimulation for the heart 400.

In some instances, pulse generators can be heart pacemakers, defibrillators, and/or neuromodulation devices. The pulse generator 402 is typically implanted subcutaneously within an implantation location or pocket in the patient's chest or abdomen. The pulse generator 402 is connected to an implantable lead 406. The lead 406 operates to convey electrical signals between the implantable pulse generator 402 and the heart 400. The lead 406 includes a flexible lead body having a proximal end portion 412 and a distal end portion 414. In various instances, the lead 406 enters the vascular system through a vascular entry site formed in the wall of the left subclavian vein. Other suitable vascular access sites may be utilized in various other instances. The lead 406 can extend through the left brachiocephalic vein and the superior vena cava such that one or more electrodes 418 disposed on the distal end portion 414 of the lead 406 can be implanted in the right atrium, right ventricle, left ventricle, or other location.

### Example 4

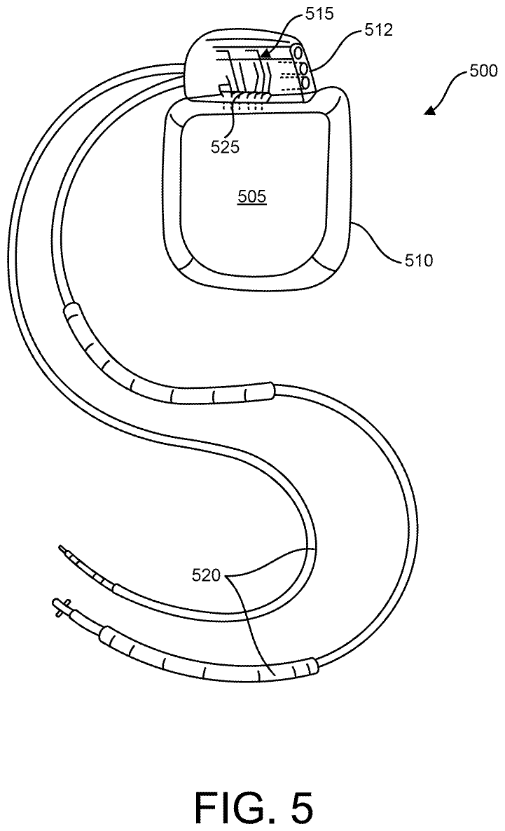

FIG. 5 provides a schematic illustration of an implantable system 500 with a partial cross-sectional view of a pulse generator 505 according to various instances. Components of the pulse generator 505 such as the outer housing, the header, and the electrical feed-thru assembly can comprise a zirconia-containing material—portions of which are metallized and comprise a barrier layer described further below.

The pulse generator 505 includes a pulse generator housing 510 including electronics, a battery power source, and a header 512 mounted on an exterior portion of the pulse generator housing 510. The pulse generator housing 510 includes a feed-thru structure, e.g., a feed-thru connector assembly 515, which operatively couples the implantable lead 520, when connected, to the electronics within the pulse generator 505. The pulse generator housing 510 includes an opening 525, according to some instances. In some instances, the housing 510 may include a plurality of openings 525.

The feed-thru connector assembly 515, in various instances, seals the opening 525 of the pulse generator 505. As such, the feed-thru connector assembly 515 hermetically seals the interior cavity of the pulse generator 505 from the external environment, e.g., sealing the interior cavity from moisture and/or biologics.

### Example Feed-Thru Assembly

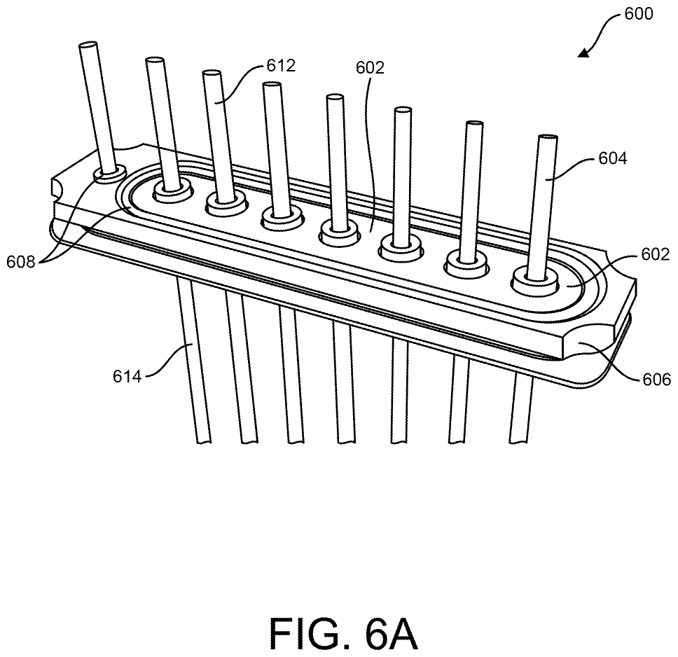

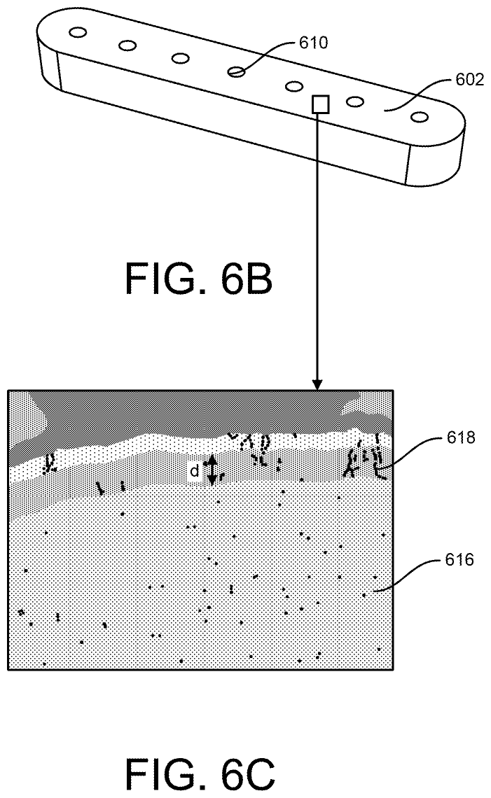

FIG. 6A is a perspective view of an exemplary feed-thru assembly 600 that can be used with the medical devices described above. FIG. 6B is a perspective front view of a base substrate 602 of the feed-thru assembly 600, and FIG. 6C is a blown-up cross-sectional view of the surface of the base substrate 602. As shown, the feed-thru assembly 600 may include a base substrate 602 connecting one or more wires 604 (e.g., conductors) and a housing component 606. The base substrate 602 may be connected to the wires 604 and housing component 606 via one or more metallic layers 608.

As shown in FIG. 6B, the base substrate 602 may include one or more openings 610 for the wires 604 to pass through and may function as a dielectric insulator in the feed-thru assembly 600. The base substrate 602 may be biocompatible such that it may be included in medical devices (e.g., different IMDs discussed in previous figures). Suitable materials used for the base substrate 602 may include zirconia based ceramics. Zirconia based ceramic materials include, for example, zirconia, stabilized zirconia, partially stabilized zirconia, tetragonal zirconia, magnesia stabilized zirconia, ceria-stabilized zirconia, yttria stabilized zirconia (“YSZ”), and calcia stabilized zirconia, as well as alumina, and titania, and the like.

In some instances, the base substrate 602 may include ranges from 0% to about 99.9% alumina, or from about 0.1% to about 95% alumina, or from about 0.2% to about 90% alumina, or from about 0.3% to about 85% alumina, or from about 0.5% to about 80% alumina, or from about 10% to about 75% alumina, or from about 20% to about 70% alumina, or a percentage of alumina encompassed within these ranges. In some instances, the base substrate 602 may include from 5% to about 99.9% zirconia, or from about 5% to about 95% zirconia, or from about 10% to about 93% zirconia, or from about 15% to about 85% zirconia, or from about 20% to about 80% zirconia, or from about 25% to about 75% zirconia, or a percentage of zirconia encompassed within these ranges. The base substrate 602 may also be a mixture of alumina and zirconia, containing about 90% alumina and about 10% zirconia, or about 75% alumina and about 25% zirconia, or about 20% alumina and about 80% zirconia, or about 0.3% alumina and about 93% zirconia. In an exemplary instance, the base substrate 602 comprise YSZ.

The base substrate 602 may have a fracture toughness up to about 15 MPa, or from about 1 to about 13 MPa, or from about 2 to about 10 MPa, or from about 3 to about 8 MPa, or from about 4 to about 7 MPa, or from about 4.5 to about 6 MPa, or a fracture toughness encompassed within these ranges. In an exemplary instance, the base substrate may have about 4.5 or about 6 MPa of fracture toughness. In some instances, the base substrate 602 has a mean grain size of from about 0.1 to about 5 μm, an E-modulus of from about 200 to about 400 GPa, a hardness from about 10 to about 20 GPa, a thermal conductivity at 20 C of about 2 to about 30 W/mK, a dielectric constant at 25 C at 1 MHz of from about 9 to about 34, a DC volume resistivity at 25 C of from about 1.00E+12 to about 5.1E+14, and/or a CTE of from about 7E(−6) to about 10E(−6)/° C.

As mentioned above, one challenge with using ceramics such as zirconia based ceramics is the ability to metallize such ceramics because tie layers used to couple metals to ceramics can consume oxygen of the underlying ceramic material. This degrades the structural integrity of the ceramic material over time.

To create a hermetic housing, the base substrate 602 (e.g., zirconia containing ceramic) may have an interface for bonding (e.g., brazing) to a housing component 606 (e.g., a titanium flange) which is then connected to the rest of the housing. One or more layers may be deposited onto the base substrate 602 to help prepare the substrate before the metallic layer 608 is deposited. For ceramic substrates, a tie layer (e.g., layer comprising titanium, chromium, or vanadium) may be deposited directly onto the ceramic substrate in order to facilitate metallization of the ceramic substrate.

However, depositing a tie layer directly onto the surface of the base substrate may—over time—break down or degrade the underlying base substrate 602 (e.g., a base ceramic substrate including zirconia), rendering it weak and prone to fracture. In certain instances, the degradation of zirconia is due to the deposited tie layer penetrating the surface of the ceramic base substrate 602 and interfering with the crystal grain structure of the base substrate 602. Zirconia (e.g., zirconium dioxide or ZrO₂) has a crystal grain structure stabilized by Zr⁴⁺ and O²⁻ ions. In some instances, the stability of the cubic crystal structure of zirconia may be enhanced at room temperature by an addition of yttrium oxide (Y₂O₃), forming yttria-stabilized zirconia (“YSZ”). When tie layers are deposited directly onto the surface of zirconia, metal from the tie layer (e.g., titanium, chromium, or vanadium) may penetrate the surface and degreade the zirconia (e.g., decreasing the oxidation state of the Zr⁴⁺ ions and binding with the O²⁻ ions in the grain structure), thus changing and weakening the otherwise stable cubic grain structure.

Instances of the present disclosure utilize the approach of applying a barrier layer 618 before depositing the tie layer 620 to help mitigate degradation of the substrate. A variety of materials may be selected for depositing the barrier layer 618. For example, materials rich in certain elements (e.g., oxygen, nitrogen, or carbon) may be used. In an exemplary instance, an oxygen rich material may be used such that metal from the tie layer 620 will react with the oxygen in the barrier layer 618 before reaching to the underlying zirconia, thus protecting the underlying zirconia substrate. Similarly, in an example where a nitrogen rich material is used in the barrier layer 618, the nitrogen may react with particles from the tie layer 620. In some instances, materials selected for the barrier layer 618 may need to withstand high temperature (e.g., a soldering or brazing temperature higher than 200 degrees C.).

As shown in FIG. 6C, the base substrate 602 may be made of a dielectric substrate 616 (e.g., zirconia based ceramic), a barrier layer 618 directly contacting the base substrate 602, and a tie layer 620 deposited on top of, and directly contacting the barrier layer 618. The barrier layer 618 can include a material comprising oxygen (e.g., aluminum oxide, titanium oxide, silicon dioxide, tantalum oxide, niobium oxide, tungsten oxide, and molybenum oxide), or a material comprising nitrogen (e.g., titanium nitride, aluminum nitride, tantalum nitride, tungsten nitride, niobium nitride, and molybdenum nitride), or a material comprising carbon (e.g., silicon carbide, tungsten carbide, aluminum carbide, tantalum carbide, and molybdenum carbide). In some instances, an oxygen rich material has an oxygen percentage of about 5% to 80%, or about 5% to 70%, or about 10% to 60%, or about 15% to 50%, or about 20% to 40%, or about 25% to 30%. In some instances, the nitrogen rich material has a nitrogen percentage of about 5% to 80%, or about 5% to 70%, or about 10% to 60%, or about 15% to 50%, or about 20% to 40%, or about 25% to 30%. In some instances, the carbon rich material has a carbon percentage of about 5% to 80%, or about 5% to 70%, or about 10% to 60%, or about 15% to 50%, or about 20% to 40%, or about 25% to 30%.

The metal of the barrier layer 618 may be any metal capable of forming an oxide or nitride or carbide. In some instances, the barrier layer 618 may have materials including metals capable of bonding with the underlying zirconia substrate and the tie layer 620. In some instances, metal included in the barrier layer 618 may have the same or relatively similar coefficient of thermal expansion (“CTE”) to the underlying substrate 616 and the tie layer 620. For example, when the tie layer 620 includes titanium, the barrier layer 618 may include titanium oxide, titanium nitride or aluminum oxide. In an exemplary instance, the tie layer 620 includes titanium, and the barrier layer 618 includes aluminum oxide.

In some instances, the barrier layer 618 may have a thickness (d) of from about 0.0005 to about 50 micron, or from about 0.001 to about 40 micron, or from about 0.05 to about 35 micron, or from about 0.1 to about 30 micron, or from about 0.5 to about 20 micron, or from about 0.6 to about 10 micron, or from about 0.7 to about 5 micron, or from about 0.8 to about 4 micron, or from about 0.9 to about 3 micron, or from about 1 to about 2 micron. The barrier layer 618 may have a maximum thickness (d) of about 50 micron. Thickness (d) may be optimized to avoid any negative impact on adhesion of the subsequent tie layers 620 to the ceramic 616. In some instances, the thickness (d) of the barrier layer 618 may be set to maintain the structural integrity and reduce stress on the tie layer 620 and/or any additional layers that may be deposited on top of the tie layer 620.

In certain instances, a desired thickness of the barrier layer 618 is dependent on the thickness of the subsequently deposited tie layer 620. For example, a thicker tie layer may dictate that a thicker barrier layer should be used such that the barrier layer has sufficient oxygen/nitrogen/carbon to be consumed by the tie layer. Additionally or alternatively, a thicker tie layer may dictate that the barrier layer should have an increased amount of oxygen/nitrogen/carbon.

In some instances, characteristics of the barrier layer may be set based on the material and specific application. Barrier layer selection may involve factors such as the zirconia based ceramic type, barrier layer type, barrier layer thickness, barrier layer composition, tie layer type, tie layer composition, and tie layer thickness. One or more of the above factors may be optimized to prevent the tie layer from reaching the ceramic and causing reduction of the ceramic substrate. As one example, the ratio of a thickness of the barrier layer to a thickness of the tie layer may be a stoichiometric ratio of the number of oxygen/nitrogen/carbon provided by each molecule of the barrier layer material and the oxygen/nitrogen/carbon included in each molecule of the tie layer material.

In some instances, where the surface of the ceramic 616 is relatively smooth, a thinner barrier layer 618 may be used. In examples where the surface of the ceramic 616 is relatively rough, a thicker barrier layer 618 may be used to conform to the surface. Depending on the roughness of the surface, the barrier layer 618 may be from about 5 nm to about 75 nm thick, or from about 6 to about 60 nm thick, or from about 7 to about 50 nm thick, or from about 8 to about 40 nm thick, or from about 9 to about 30 nm thick, or from about 10 to about 20 nm thick. In some examples where the surface is relatively smooth, the barrier layer 618 may be less than 10 nm thick, or about 5 nm thick. In some examples, to work with a relatively rough surface, the barrier layer 618 may be about 75 nm thick.

Once the barrier layer 618 is applied, additional top layers for metallization (e.g., the tie layer 620 and the metal layer) can be applied with reduced risk of ceramic degradation. The top layers will then allow for brazing, bonding, conductive, optical, and protective layers needed in device designs for ceramic based components used in IMD or ablation devices.

Methods of depositing the barrier layer 618 include sputtering, flame spray, thermal evaporation, or any suitable physical vapor deposition method. In some instances, a barrier layer 618 containing titanium nitride may be deposited using thermal evaporation. In some instances, a barrier layer 618 containing aluminum oxide may be deposited by sputtering.

Referring now back to FIG. 6A, the feed-thru assembly 600 can include a conductor disposed at least partially within the opening 610. As shown in FIG. 6A, for example, the conductor can be a wire 604 disposed within the opening 610. Other examples of suitable conductor types, in some instances, include a ribbon, a filament, and any rod with a polygonal shaped cross-section, such as a triangular or a hexagonal cross-section.

The wire 604 can be positioned within the opening 610. In some instances, the wire 604 is positioned such that a portion of the wire 604 is located external to an IMD housing and a portion of the wire 604 is located internally within the IMD housing. For example, the wire 604 may be oriented such that a proximal end 612 of the wire 604 extends outwardly from the opening 610 and a distal end 614 extends into an inner cavity of an IMD housing. The feed-thru assembly 600 allows an implantable lead (e.g., the implantable lead 520) to be coupled to electronics and a source battery within an IMD (e.g., the pulse generator 505 shown in FIG. 5) through the wire 604.

The wire 604 may be also described as a feed-thru wire, a pin or a conductor. In some instances, the wire 604 may be straight (that is, the distal and proximate ends of the wire 604 may axially align). The wire 604 may be operatively connected either directly or indirectly to electronics (e.g., a printed circuit board or other electronic components) within an IMD housing. The wire 604 may be fixed directly to the printed circuit board or the other electronic components. In some instances, the wire 604 may be soldered, wire-bonded or welded to the printed circuit board or the other electronic components.

The wire 604 is made of a metallic material in various instances. For example, in some instances, the wire 604 is made of titanium. The wire 604 may be formed of any suitable material, for example, titanium, tantalum, tungsten, platinum, palladium, stainless steels (e.g., SS316), nickel-cobalt-chromium-molybdenum alloys such as MP35N, MP35N/silver drawn braze strand, MP35N/silver drawn filled tube, nitinol, cobalt-chromium-nickel alloy (e.g., elgiloy), cobalt-chromium-nickel-molybdenum-iron alloys or combinations thereof. Other exemplary materials for the wire 604 include, but are not limited to, platinum iridium (Ptlr), palladium iridium (Pdlr), silver (Ag), gold (Au) or combinations thereof.

The housing component 606 may be made of the same material as the housing. In some instances, the housing component 606 is made of titanium, titanium alloy, MP35N, stainless steel or combinations thereof. In an exemplary instance, the housing component 606 may be a titanium flange for connecting to an IMD housing. As shown, the metallic layers 608 are coupled to the tie layer 620. In some instances, the metallic layers 608 may be a brazing material (e.g., copper, nickel, silver, aluminum, gold).

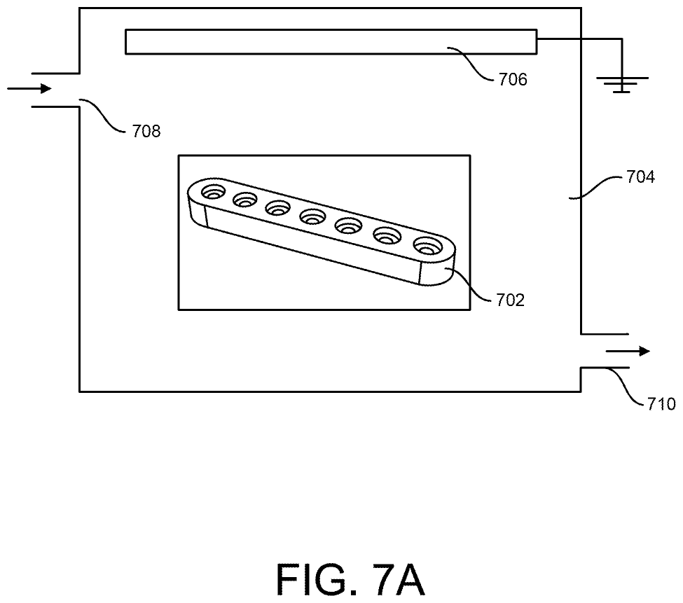

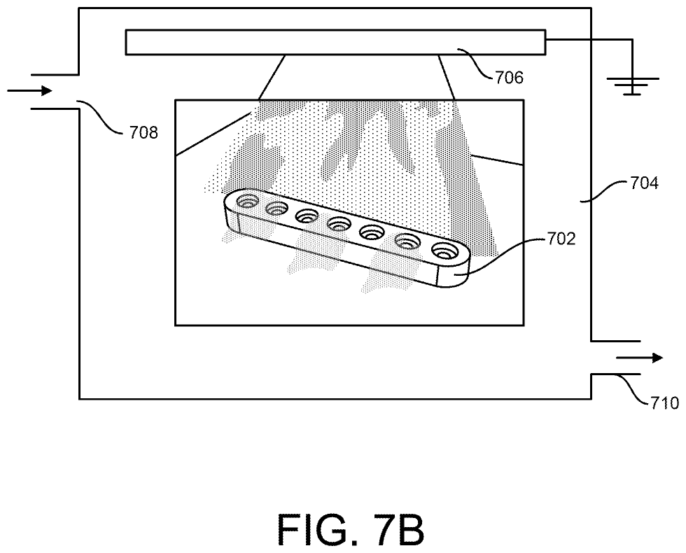

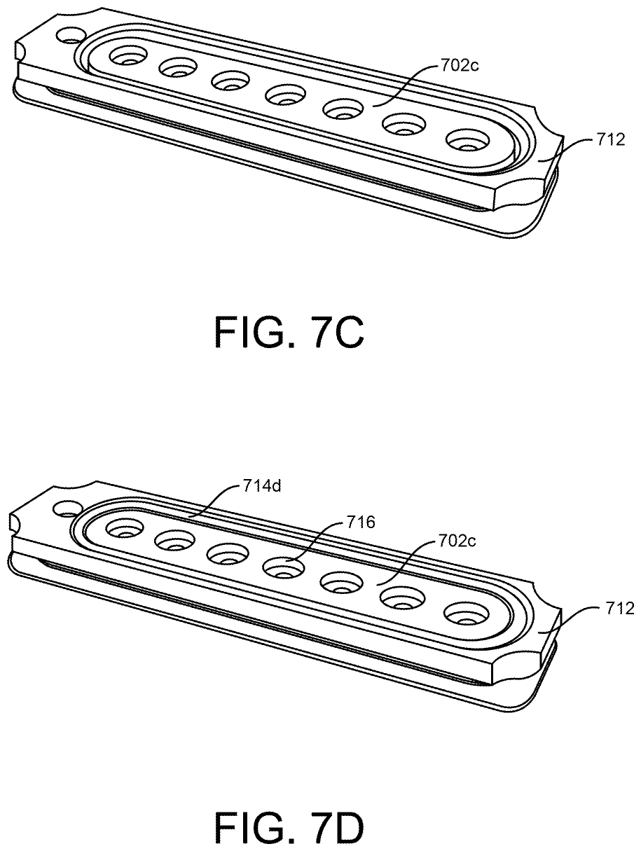

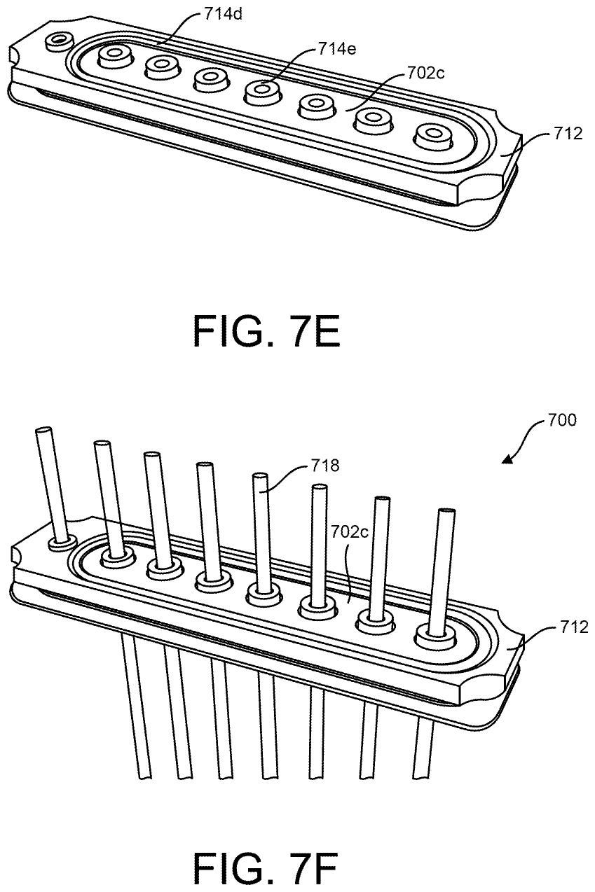

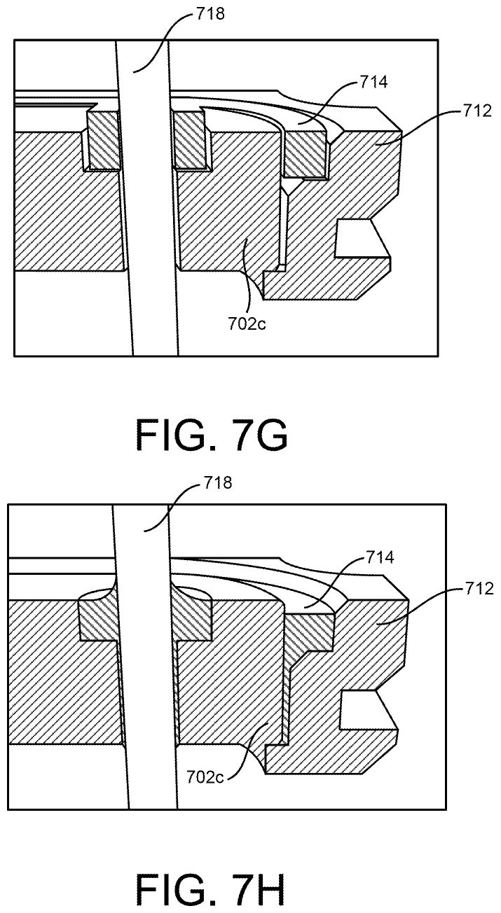

FIGS. 7A-7H are schematic illustrations of an assembly process of an exemplary feed-thru assembly, in accordance with instances of the present disclosure. The assembly process shown in FIGS. 7A-7H are examples of the various features of methods to metalize a substrate for making a feed-thru assembly, although the combination of those illustrated features is clearly within the scope of invention, that example and its illustration is not meant to suggest the inventive concepts provided herein are limited from fewer features, additional features, or alternative features to one or more of those features shown in FIGS. 7A-7H.

In addition, one or more steps of the below process may be optional and/or may be modified by one or more steps of other instances described herein. Additionally, one or more steps of other instances described herein may be added to the process.

As shown, a base substrate 702 is placed in a sputtering chamber 704. The base substrate 702 may be a dielectric insulator for a component of a medical device such as a housing or a feed-thru assembly through which an electrical lead passes. The substrate 702 may be a dielectric ceramic substrate selected from a group of zirconia, stabilized zirconia, partially stabilized zirconia, tetragonal zirconia, magnesia stabilized zirconia, ceria-stabilized zirconia, yttria stabilized zirconia, and calcia stabilized zirconia, as well as alumina, titania, and the like. The sputtering chamber may include a sputtering target 706, a gas inlet 708, and a gas outlet 710.

A barrier layer may be deposited (e.g., sputtered as shown in FIG. 7B) onto the substrate 702 first, then a tie layer is deposited onto the barrier layer. The barrier layer 618 can include a material comprising oxygen (e.g., aluminum oxide, titanium oxide, silicon dioxide, tantalum oxide, niobium oxide, tungsten oxide, and molybenum oxide), or a material comprising nitrogen (e.g., titanium nitride, aluminum nitride, tantalum nitride, tungsten nitride, niobium nitride, and molybdenum nitride) or a material comprising carbon (e.g., silicon carbide, tungsten carbide, aluminum carbide, tantalum carbide, and molybdenum carbide). In some instances, the oxygen rich material has an oxygen percentage of about 5% to 80%, or about 5% to 70%, or about 10% to 60%, or about 15% to 50%, or about 20% to 40%, or about 25% to 30%. In some instances, the nitrogen rich material has a nitrogen percentage of about 5% to 80%, or about 5% to 70%, or about 10% to 60%, or about 15% to 50%, or about 20% to 40%, or about 25% to 30%. In some instances, the carbon rich material has a carbon percentage of about 5% to 80%, or about 5% to 70%, or about 10% to 60%, or about 15% to 50%, or about 20% to 40%, or about 25% to 30%. The tie layer may include titanium, chromium, molybdenum, niobium, tungsten, tantalum, or vanadium.

After the substrate 702 is coated with the layers, as shown in FIG. 7C, the coated substrate 702c may then be removed from the sputtering chamber 704, and placed within a housing component 712. The housing component 712 may be shaped and configured to later attach to a housing of an IMD. A first top metallizing layer 714d may then be deposited onto the tie layer, and in a space in between the coated substrate 702c and the housing component 712 (see FIG. 7D). The top metalizing layer 714d may allow for brazing or bonding the dielectric substrate 702 to the housing component 712.

In some instances, the substrate 702 may include one or more openings 716. A second top metallizing layer 714e may be deposited onto the tie layer of the substrate 702 inside the one or more openings 716 (see FIG. 7E). In some instances, the metalizing layer 714 may include a precious metal, such as gold, to braze the wires 718 to the substrate 702 that may be a dielectric insulator. In some instances, the substrate 702 may also be brazed to a titanium or niobium ferrule with the precious metal. One or more electrical leads or wires 718 may be subsequently inserted through each of the openings 716 of the substrate 702 (see FIG. 7F).

FIG. 7G-7H are partial cross-sectional views of the feed-thru assembly 700 shown in FIG. 7F. As the assembly 700 is heated to a brazing temperature, the metalizing layers 714 bonds the wire 718 to the substrate 702, as well as the substrate 702 to the housing component 712, thus locking in these components to form a hermetic seal.

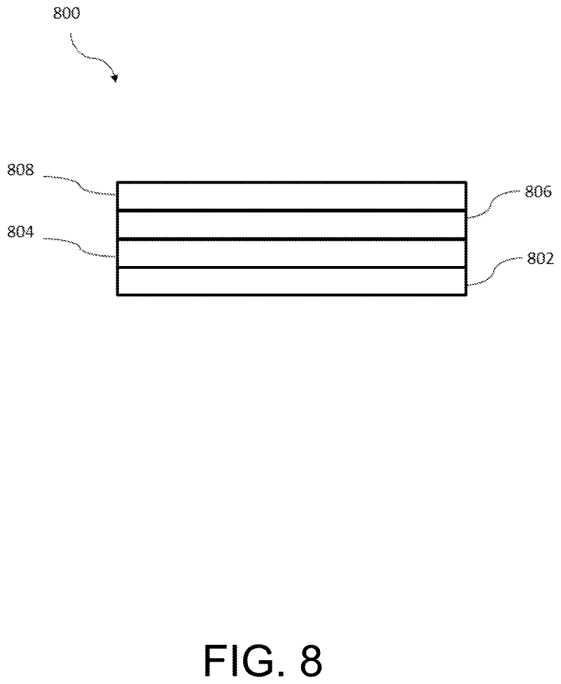

FIG. 8 shows a schematic of a four-layer structure 800. The bottom layer is a substrate 802 such as the substrate examples discussed above. For example, in certain instances, the substrate 802 comprises a ceramic material that comprises zirconia. Directly above and directly contacting the substrate 802 is a barrier layer 804 such as the barrier layer examples discussed above. For example, the barrier layer 804 can comprise a material that comprises oxygen, nitrogen, and/or carbon to protect the substrate 802 from oxygen-hungry, nitrogen-hungry, and/or carbon-hungry tie layer materials. Directly above and directly contacting the barrier layer 804 is a tie layer 806 such as the tie layer examples discussed above. For example, the tie layer 806 can comprise a material that can bond with a metal layer. Directly above and directly contacting the tie layer 806 is a metal layer 808 such as the metal layer examples discussed above. For example, the metal layer 808 can comprise a metal. Using the structure 800, a ceramic substrate 802 can be metalized. The structure 800 can be incorporated into many different applications including, but not limited to, the medical device applications described above.

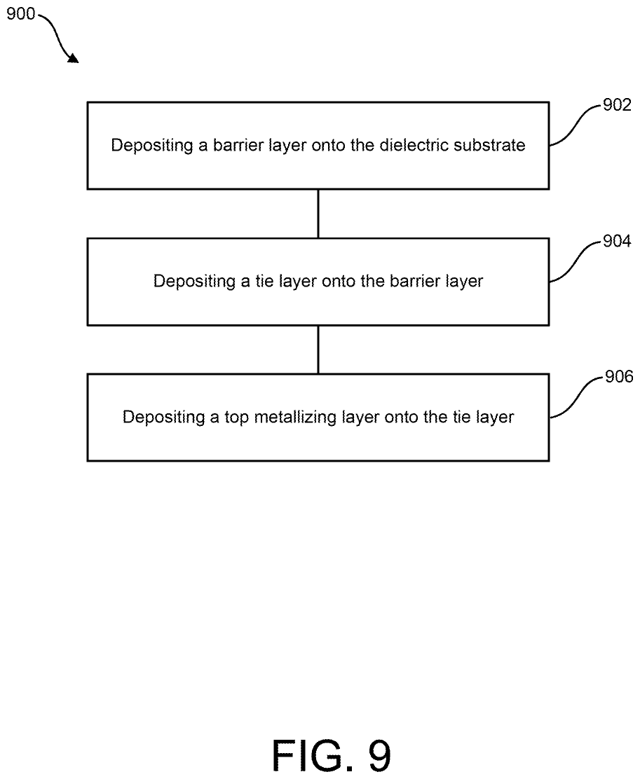

FIG. 9 is a flowchart illustrating a method 900 of metallizing a ceramic substrate (e.g., a zirconia based material such as zirconia, stabilized zirconia, partially stabilized zirconia, tetragonal zirconia, magnesia stabilized zirconia, ceria-stabilized zirconia, yttria stabilized zirconia, and calcia stabilized zirconia, as well as alumina, and titania, and the like).

At step 902, the method 900 includes depositing a barrier layer onto the substrate. At step 904, the method 900 includes depositing a tie layer onto the barrier layer. The tie layer may include titanium, chromium, or vanadium. The layers may be deposited by using physical vapor deposition methods such as sputtering, thermal evaporation, or flame spray.

At step 906, the method 900 includes depositing a top metallizing layer onto the tie layer. The top metallizing layer allows for brazing or bonding the dielectric substrate.

Various modifications and additions can be made to the exemplary instances discussed without departing from the scope of the present disclosure. For example, while the instances described above refer to particular features, the scope of this disclosure also includes instances having different combinations of features and instances that do not include all of the described features. Accordingly, the scope of the present disclosure is intended to embrace all such alternatives, modifications, and variations as fall within the scope of the claims, together with all equivalents thereof.

We claim:

1. A method of metallizing a ceramic substrate, the method comprising:
depositing a barrier layer onto the ceramic substrate;
depositing a tie layer onto the barrier layer; and
depositing a metal layer onto the tie layer to metallize the ceramic substrate,
wherein the barrier layer comprises an oxygen rich material, a nitrogen rich material, or a carbon rich material.

2. The method of claim 1, wherein the dielectric substrate comprises zirconia.

3. The method claim 1, wherein the dielectric substrate is selected from a group consisting of zirconia, stabilized zirconia, partially stabilized zirconia, tetragonal zirconia, magnesia stabilized zirconia, ceria-stabilized zirconia, yttria stabilized zirconia, and calcia stabilized zirconia, as well as alumina, titania, and the like.

4. The method of claim 1, wherein the oxygen rich material is selected from a group consisting of aluminum oxide, titanium oxide, silicon dioxide, tantalum oxide, niobium oxide, tungsten oxide, and molybenum oxide.

5. The method of claim 1, wherein the oxygen rich material has an oxygen percentage of 5% to 80%.

6. The method of claim 1, wherein the nitrogen rich material is selected from a group consisting of titanium nitride, aluminum nitride, tantalum nitride, tungsten nitride, niobium nitride, and molybdenum nitride.

7. The method of claim 1, wherein the nitrogen rich material has a nitrogen percentage of 5% to 80%.

8. The method of claim 1, wherein the carbon rich material is selected from a group consisting of silicon carbide, tungsten carbide, aluminum carbide, tantalum carbide, and molybdenum carbide.

9. The method of claim 1, wherein the carbon rich material has a carbon percentage of 5% to 80%.

10. The method of claim 1, wherein the tie layer comprises titanium, chromium, molybdenum, niobium, tungsten, tantalum, or vanadium.

11. The method of claim 1, wherein the barrier layer is deposited onto the dielectric substrate by sputtering, thermal evaporation, or flame spray.

12. The method of claim 1, wherein the metal layer is a brazing material.

13. The method of claim 1, wherein a thickness of the barrier layer is 0.0005 to 50 microns.

14. A metallized ceramic substrate comprising:
a base ceramic substrate comprising zirconium;
a barrier layer directly contacting the base substrate;
a tie layer directly contacting the barrier layer; and
a metallic layer coupled to the tie layer.

15. The ceramic substrate of claim 14, wherein the metallic layer is a brazing material.

16. An implantable medical device comprising:
a feed-through assembly with a metallized ceramic substrate that includes:
a ceramic substrate comprising zirconium;
a barrier layer directly contacting the base substrate;
a tie layer directly contacting the barrier layer; and
a metal layer coupled to the tie layer.

17. The implantable medical device of claim 16, further comprising:
one or more wires bonded to the metallized ceramic substrate.

18. The implantable medical device of claim 16, wherein the barrier layer comprises aluminum oxide.

19. The implantable medical device of claim 16, wherein the substrate is yttria stabilized zirconia.

20. The implantable medical device of claim 16, wherein the tie layer comprises titanium.
